(19) United States
(12) Patent Application Publication
(10) Pub. No.: US 2024/0057634 A1
(43) Pub. Date: Feb. 22, 2024

(54) OAT PROTEIN COMPOSITION OF HIGH SOLUBILITY

(71) Applicant: ROQUETTE FRERES, Lestrem (FR)

(72) Inventors: Leon ZHOU, Geneva, IL (US); Ron CHENG, Geneva, IL (US); Kerry CAMPBELL, Geneva, IL (US)

(21) Appl. No.: 18/259,951

(22) PCT Filed: Jan. 4, 2022
(86) PCT No.: PCT/EP2022/025003

§ 371 Date: Jun. 29, 2023

(30) Foreign Application Priority Data
Jan. 4, 2021 (EP) 21305005.7

(51) Int. Cl. A23J 1/12; A23J 3/14

(52) U.S. Cl. CPC A23J 1/12; A23J 3/14; A23J 3/26

(57) ABSTRACT

The invention pertains to the field of oat protein compositions and production method thereof. In particular, the present invention also concerns oat protein compositions having high solubility, which are not chemically substituted. The present invention also concerns methods of production of these oat protein compositions.

### TECHNICAL FIELD

The invention pertains to the field of oat protein compositions and production method thereof. In particular, the present invention also concerns oat protein compositions having high solubility, which are not chemically substituted. The present invention also concerns methods of production of these oat protein compositions.

### BACKGROUND OF THE INVENTION

Oats are a well-known source of a wide variety of useful products. Examples of such products are flour, starch, protein isolate and concentrate, protein-enriched flour, bran, gum and oil. Oats contain globulin as the major storage protein (around 75% by weight), other proteins being mainly prolamins and albumins. At native state, oat globulins are soluble in salt solutions whereas oat prolamins are soluble in aqueous ethanol and oat albumins are soluble in water. Globulins are considered as high molecular weight proteins, typically above 50 kDa, whereas prolamins and albumins are considered both having low molecular weight, generally below 50 kDa.

The solubility of oat protein compositions is generally considered as very low compared to other leguminous, tuber or other cereal proteins. It is especially limited around neutral pH, which is a typical pH range for most food products.

For example, in the field of beverages, it is very important to have improved solubility at this range of pH. Indeed, ready to drink beverages having neutral pH are today of most importance in terms of market size. It is needed that the protein used in the beverage allows to obtain beverages having a texture that is stable over time.

This issue is one important reason that explains why oat protein compositions have not significantly entered the market of protein isolates: in 2020, the inventors are aware of only one oat protein composition on the market, that is today commercialized under the brand name PROATEIN® by Lantmannen Oats (protein content 50-60%). PROATEIN® product is an oat protein concentrate that presents a low solubility at neutral pH, lower than 20% and typically contains 16-19% oil.

Solubility is critical for use of protein in food applications, as soluble proteins provide homogenous dispersability in colloidal systems and enhance the interfacial properties. This is even more important when the compositions have an increased amount of protein in the oat protein compositions, e.g. to have a content of protein of 70% or above.

To modify the properties of oat protein isolates, for example gel properties, different techniques have been used.

Low temperature or medium temperature heat treatments of alkaline oat protein suspensions have been described in the literature. However none of these documents describe the improvement of solubility of the oat protein at the pH range 4-7. Ma et al., (J. Agric. Food Chem., 38 (1990)) has already disclosed an alkaline treatment at a maximum temperature of 55° C. for several days. Besides the fact that these kind of treatments are not favorable in terms of bacteriology, the solubility of the oat protein at neutral pH is not disclosed in this document. Medium temperature heat treatments (100° C.) at alkaline pH of salt-containing (and other structure “perturbants” containing) oat protein compositions are also described in two papers (Ma et al., J. Agric. Food Chem., 36 (1988) and Ma et al., Spectroscopy, 17 (2003)). The solubility of the protein at neutral pH is not increased in these documents and, on the opposite, a solid gel structure appears after this medium temperature heat treatment.

To transform the insoluble oat protein compositions into more soluble compositions, different techniques have been used.

A first possibility is to substitute chemically the oat protein. The use of succinylation or acylation (e.g. acetylation) are already known to achieve the increase of solubility. For example, Mirmoghtadaie et al., Food Chemistry 114 (2009), have demonstrated that by succinylation, the nitrogen solubility index of an oat protein isolate was increased from 22.9% to 86.8%. Succinylation and acetylation involves reacting food proteins with succinic anhydride and acetic anhydride. The use of these chemicals in food presents regulatory challenges and is not considered a “clean label” process.

Another option is to use enzymes to increase the solubility of oat protein compositions.

One enzyme sub-option is to use an endoprotease to hydrolyze the protein and decrease the molecular weight to make it more soluble. Wang et al. in Food Biophysics (October 2014) have described the endoprotease (alcalase) hydrolysis of oat protein isolates. The issue of using hydrolysis is that proteins obtained can have bad taste if there is an important degree of hydrolysis. The hydrolysis also decreases molecular weight protein, and this can be unfavorable for some applications, for example when it is needed that the protein gives mouthfeel to a beverage.

Another enzyme sub-option is to use glutaminase. Glutaminase is an amidohydrolase which has the ability to enzymatically deamidate the protein and generates glutamate from glutamine. Jiang et al. have demonstrated that, without significant changes in the molecular weight of the protein chains, an oat protein isolate can only have increased solubility Nitrogen Solubility Index (NSI) (around 90%) when the deamidation degree of the oat protein is very important. In this paper, when the oat protein is deamidated at 42% or lower, there is no significant modification of the solubility of the oat protein. Deamidation is considered a form of protein degradation and results in the loss of nitrogen in the form of ammonia when asparagine or glutamine are converted to aspartate and glutamate. Deamidation results in a reduced protein content and reduced nutritional quality so it may be helpful to limit, or even to prevent completely the deamidation of a protein.

In a different approach, Brückner-Gihmann et al., European Food Research and Technology, Vol. 244, no 12 (2018) describes the production of an oat protein isolate using an oat protein concentrate as a starting material. The process of obtaining this oat protein isolate uses a step of alkaline extraction of this concentrate, a step of separation of the soluble protein into the supernatant from other insoluble components. In this process most of the globulins, which generally represent around 75% of weight of the proteins in the flour, are then eliminated with the starch and insoluble fibers. The obtained protein isolate presents a solubility NSI of around 73%, but comprises around 90% of proteins having a molecular weight below 50 kDa, i.e. the molecular weight representative of prolamins and albumins. The issue is that these proteins have no ability to provide mouthfeel to a beverage product and presents too much foaming properties. From a commercial processor's perspective, the loss of the globulin protein fraction represents a financially prohibitive loss of protein yield.

It appears from the above that there is still a need to make available high solubility oat protein compositions of high molecular weight that are not significantly chemically substituted.

### DESCRIPTION OF THE INVENTION

It is one of the achievements of the invention to provide new oat protein compositions presenting these characteristics. Indeed, when investigating the manufacturing of oat protein isolates of high molecular weight, the inventors have found new processes to improve the solubility of these, without using process steps that chemically substitute the proteins significantly. Another embodiment of the invention thus concerns processes of manufacturing soluble oat protein composition of high molecular weight.

More precisely, the invention concerns an oat protein composition having a protein content higher than 50% wherein:
the deamidation degree of the protein is below 40%,
the protein is not substantially chemically substituted,
the solubility of the composition at pH 7 is above 50%,
and the molecular weight Mw of the protein composition is above 25000 g/mol, preferably above 30000 g/mol.

These new oat protein compositions are of high interest for food applications and especially for beverages.

One other object is a process of manufacturing an oat protein composition having a solubility at pH 7 above 50% comprising:
a step of providing a suspension of an oat proteic product, the oat proteic product having a protein content higher than 50% and a molecular weight Mw above 25000 g/mol, preferably above 30000 g/mol,
a treatment step of this oat protein suspension, the treatment being chosen from an ultrasonication treatment step and an heat treatment step wherein, in the case of the heat treatment of the suspension:
   the pH of the suspension is between 8 and 11,
   the temperature of the suspension is between 120 and 180° C.,
      wherein no enzymatic treatment step using endoprotease is done after the treatment step of the oat proteic product suspension.

The inventors have developed this new process that increases the solubility of oat protein without substantially chemically substituting, hydrolyzing or deamidating the protein structure. This process comprises a specific heat treatment step at high temperature and/or ultrasonication treatment step. Regarding the heat treatment, the inventors have surprisingly observed that it allows to obtain higher solubility at neutral pH whereas, as discussed above, literature describe that these heat treatments allow the formation of protein gels. These gels are agglomerated non-soluble protein structures that are formed when cooled down at room temperature. Regarding the ultrasonication step, similar step is described in the paper Wang et al. presented previously. However, the process of Wang et al. comprises a subsequent endoprotease treatment of the oat protein using alcalase. Furthermore, Wang et al. have demonstrated that a preliminary ultrasound treatment of oat protein isolate, before an alcalase treatment, allows the alcalase treatment to be more efficient. Authors made the hypothesis that hydrophobic bonds inside the oat protein isolate are delocalized outside thanks to this ultrasound treatment. The alcalase can be activated in these new sites, and this explains higher efficiency of proteolytic treatment. It was then expected that using such ultrasonic treatment on oat protein isolate would increase the number of hydrophobic bonds outside of the protein and would decrease the solubility of the oat protein isolate. This phenomenon of insolubilization is generally observed when applying ultrasonication to plant proteins: for example, the sonication treatments of lupine flour before isolation decreases protein isolate solubility (Aguilar-Acosta et al., Biomolecules, 10, 292 (2020)). Surprisingly, a process comprising sonication treatment step of an oat protein suspension allows to obtain an oat protein composition of high molecular weight that are not significantly chemically substituted that presents high solubility.

By “oat protein composition”, it is meant a composition comprising essentially oat protein as the only source of protein. In other terms, the oat protein composition does not comprise any protein that comes from another origin than oat.

“Oat” in the present application must be understood as a cereal plant belonging to the botanical genus Avena. This genus can be divided in wild and cultivated species which have been cultivated for thousands of years as a food source for humans and livestock. The cultivated species contain:
Avena sativa—the most cultivated specie, commonly referred to as “oats”.
Avena abyssinica—the Ethiopian oat, native to Ethiopia, Eritrea, and Djibouti; naturalized in Yemen and in Saudi Arabia
Avena byzantina, a minor crop in Greece and Middle East; introduced in Spain, Algeria, India, New Zealand, South America, etc.
Avena nuda—the naked oat or hulless oat, which plays the same role in Europe as does A. abyssinicain Ethiopia. It is sometimes included in A. sativa and was widely grown in Europe before the latter replaced it. As its nutrient content is somewhat better than that of the common oat, A. nuda has increased in significance in recent years, especially in organic farming.
Avena strigosa—the lopsided oat, bristle oat, or black oat, grown for fodder in parts of Western Europe and Brazil.

In a preferred embodiment, oat protein composition is a protein concentrate, or a protein isolate. The oat protein composition can thus have around 50% by weight of protein or above, based on dry matter based on the total dry weight of the oat protein composition, for example from around 55 to 90%.

In the present application, “protein concentrate” must be understood as an oat protein composition which contains from 50% to 70%, by weight of protein (i.e. oat protein) on dry matter based on the total dry weight of the oat protein composition.

In the present application, “protein isolate” must be understood as an oat protein composition which contains more than 70%, generally more than 75%, preferably more than 80% by weight of protein on dry matter based on the total dry weight of the oat protein composition. The protein isolate can comprise less than 95%, generally less than 90% of protein on dry matter based on the total dry weight of the oat protein composition.

Various protocols can be used from prior art in order to quantify the protein content. In the present application, a preferred method to quantify the protein content consists of 1) determining the nitrogen content in the composition and 2) multiplying the nitrogen content by 6.25 factor (which represent the average quantity of nitrogen in protein). The nitrogen content can be determined by any suitable method in the art, such as the Kjeldhal method or by using a combustion analyzer. Preferably, the nitrogen content is determined by a combustion analyzer.

In the present application “protein” must be understood as molecules, consisting of one or more long chains of amino-acid residues. In the present application, proteins are of high molecular weight. These proteins can be present in different concentrations, including protein isolates or protein concentrates. Oats are the only cereal containing avenalin as globulin or legume-like protein, as the major storage protein (80% by weight). Native globulins are generally characterized by their solubility in dilute saline as opposed to the more typical cereal proteins, such as gluten and zein which is a prolamine. The minor protein of oat is the prolamine which is called avenin. The composition can comprise such kind of proteins, or amino acids, as long as it comprises an amount sufficiently low that the molecular weight Mw of the protein composition is above 25000 g/mol, preferably above 30000 g/mol.

In the present application, “the molecular weight Mw” must be understood as the weight average molecular weight. Preferably, the oat protein composition of the invention has a molecular weight Mw of the protein composition is above 40000 g/mol, for example more than 45000 g/mol or more than 50000 g/mol.

The protein molecular weight distribution can be determined using Size Exclusion Chromatography. To do so, it is possible to use the following method which was used to determine molecular weight in the example section. Samples can be dissolved in 200 mM phosphate buffer, pH=7.6, vortexed for 1 minute initially and 10 minutes later and stored at 4 C over-night. The solutions are centrifuged at 7000 g for 10 minutes, the supernatant is measured for soluble protein content the next day, and the samples are diluted to 10 mg/mL with phosphate buffer. The samples are chromatographed using 2 SEC columns (400 and 300 Agilent Advanced Bio SEC Column, 5000-1,250,000 MW Range) in sequence using phosphate buffer, pH=7.6 as the mobile phase at 0.5 mL/minute. The detection is a UV=280 nm. Several protein molecular weight standards going from 14300 to 669000 Da (Lysozyme, Carbonic Anhydrase, BSA, HSA, B-Amylase, Apoferritin, Thyroglobulin) are analyzed to identify the retention time and calibrate the chromatography apparatus. For sample analysis, chromatograms peak or peak apex (group) is determined along with the range of the peak (start and end) and the molecular weight is determined for the range and peak apex. The percent of molecular weight can be determined, for example, for: >300 kDa, 300 kDa to 50 kDa, 50 KDa to 10 KDa and <10 kDa.

Preferably, the oat protein composition comprises, based on the total weight of proteins in the composition at least 14% of proteins having a molecular weight of at least 50000 g/mol.

Preferably, the oat protein composition comprises, based on the total weight of proteins in the composition:
from 0.5 to 30% of proteins having a molecular weight of 300000 g/mol and more, advantageously from 1 to 20%, more preferably from 5 to 15%,
from 10 to 75% of proteins having a molecular weight of between 50000 g/mol and 300000 g/mol, advantageously from 30 to 75%, more preferably from 20 to 70% or from 45 to 65%,
from 10 to 50% of proteins having a molecular weight of between 10000 g/mol and 50000 g/mol, advantageously from 25 to 45%,
from 0.5 to 40% of proteins having a molecular weight of 10000 g/mol and less, advantageously from 0.5 to 20%, even more preferably from 1 to 10%, the sum making 100%.

Preferably, the oat protein composition comprises, based on the total weight of proteins in the composition:
from 0.5 to 30% of proteins having a molecular weight of 300000 g/mol and more, advantageously from 5 to 15%,
from 30 to 75% of proteins having a molecular weight of between 50000 g/mol and 300000 g/mol, advantageously from 45 to 65%,
from 10 to 50% of proteins having a molecular weight of between 10000 g/mol and 50000 g/mol, advantageously from 25 to 45%,
from 0.5 to 20% of proteins having a molecular weight of 10000 g/mol and less, advantageously from 1 to 10%,
   the sum making 100%.

In the composition of the invention, the protein can be not substantially chemically substituted, i.e. the end functions of the protein have not undergone chemical reaction with substitution reactors, such as succinic or acetic anhydride. In the composition of the invention, the protein can present a deamidation degree below 40%. The deamidation degree of the protein is advantageously below 20%, for example below 15%, or below 10%, or below 5% or around 0%. The deamidation degree can be determined by any known methods. One method can consist in deamidating totally a non-deamidated oat protein, determine the ammonia content generated during total deamidation and compare with ammonia content in the sample of the oat protein to analyze. Total deamidation of a non-deamidated oat protein can be done by dissolving 0.5 to 3 grams of material into 2 N HCl aqueous solution in a round bottom flask, allowing to hydrate by mixing in a concentration of 5 to 10 grams of protein per liter, heating to a boil and refluxed for 2 hours with constant mixing, then cooling, adjusting to a pH of 7.8 with an 4N NaOH aqueous solution, measuring the volume obtained, centrifuging the solution in a microfuge at 13,000 rpm, and determining the ammonia content of 0.1 mL of material. Determination of ammonia content in 0.1 mL can be done by using Megazyme procedure: diluting the sample in buffer (oxoglutarate, pH=8) with the addition of reduced form of Nicotinamide adenine dinucleotide phosphate (NADPH), after 2 minutes of equilibration reading absorbance at 340 nm as a baseline in a spectrophotometer, adding Glutamate Dehydrogenase and allowing to react for 5 minutes to convert oxoglutarate to lactic acid and NADPH (which is UV detectable at 340 nm) to NADP⁺. The drop in UV absorbance at 340 nm from initial baseline reading is used in the calculation to calculate ammonia (mg/mL). The content of ammonia is then calculated using the extinction coefficient of NADPH. The final NH₃ concentration (mg/mL) is divided by the protein concentration (mg protein/mL) of the sample preparation and converted to a percentage of ammonia in the sample. The deamidation degree in percentage is determined by dividing the content of ammonia in the sample with the ammonia content in the sample generated when proceeding to the total deamidation of a non-deamidated oat protein.

In the present application, the “solubility of the oat protein composition” means the content of soluble matter of the composition based on the total dry matter of the oat protein composition when determined at pH 7, generally at 20° C. Preferably, the oat protein composition has a solubility at pH 7 above 50%, advantageously above 60%, more advantageously above 70%, preferably above 75%, more preferably above 80%. 6. The oat protein composition can also have a solubility at pH 4, generally at 20° C., above 10%, advantageously above 15%, more advantageously above 20%. These solubilities can be determined by any known methods. Depending on whether the oat protein composition is in a solid or liquid form, solubility can be determined in a different manner. Preferred methods for both solid and liquid are indicated in the examples section.

The oat protein composition can comprise an extractable lipid content below 20% by weight on dry matter based on the total dry weight of the oat protein composition. In the present application “extractable lipid” must be understood as molecules that are soluble in nonpolar solvents for example petroleum ether, i.e. extractable lipids. Lipids include fatty acids, waxes, sterols, fat-soluble vitamins (such as vitamins A, D, E, and K), monoglycerides, diglycerides and triglycerides. Oats, after corn, have the highest lipid content of all the cereals, i.e. greater than 6%, sometimes greater than 10% by weight for some oats, in comparison to about 2-3% by weight for wheat and most other cereals.

According to an embodiment of the invention, the oat protein composition comprises an extractable lipid content below 10% by weight on dry matter based on the total dry weight of the oat protein composition, advantageously below 9%, more advantageously below 8%, even more advantageously below 7%, preferentially below 6%. The oat protein composition of the invention may comprise an extractable lipid content above 1% by weight on dry matter based on the total dry weight of the oat protein composition, for example more than 2%. The oat protein composition of the invention comprises a total lipid content below 10% by weight on dry matter based on the total dry weight of the oat protein composition, advantageously below 9%, more advantageously below 8%. The oat protein composition of the invention may comprise a total lipid content above 1% by weight on dry matter based on the total dry weight of the oat protein composition, for example more than 2%.

The total lipid content used for the invention is acid hydrolysis using AOAC 996.06 method, while extractable lipid is measured by Soxhlet method using petroleum ether using AOAC 963.15 protocol.

The oat protein composition can comprise other components than protein and lipid such as starch, starch hydrolysate, fibers or minerals, the total content of these other components being generally in a quantity lower than 40%, more generally lower than 30%, more generally lower than 20%, based on the total dry substance of the composition.

The oat protein composition can also comprise from 0 to 40% by weight of starch based on the total dry weight of the oat protein composition, generally from 2 to 25%. Starch content of the composition can be determined using AOAC Official Method 996.11, Starch (Total) in Cereal Products, and more particularly using the method of the booklet Megazyme, Total starch assay procedure (amyloglucosidase/α-amylase method) K-TSTA-50A/K-TSTA-50A 11/20, AOAC 996.11.

The oat protein composition can also comprise from 0 to 40% of starch hydrolysate based on the total dry weight of the oat protein composition, generally from 2 to 25%. Starch hydrolysate should be understood as “maltodextrins” and this content can be determined using the booklet Megazyme cited above.

The oat protein composition can comprise a soluble fiber content going below 15% by weight on dry matter based on the total dry weight of the oat protein composition.

The oat protein composition can comprise an insoluble fiber content going below 15% by weight on dry matter based on the total dry weight of the oat protein composition.

In the present application, soluble fiber content, insoluble fiber content, fiber content (which includes the total of soluble and insoluble fiber contents) can be determined using AOAC Official Method 2017.16, Total Dietary Fiber in Foods and Food Ingredients. By soluble fibers, it is meant to be fibers soluble in ethanol as described in this method. One of the dietary fibers generally present in the composition is beta-glucans.

In the present application, “dry matter” must be understood as the relative percentage by weight of solids based on total weight of the sample. Every well-known method can be used but desiccation method, which consists of estimating quantity of water by heating a known quantity of sample, is preferred.

In an embodiment, the oat protein composition is in a powder form, the powder having advantageously a mean particle size d-50 greater than 10 microns, more advantageously greater than 20 microns, preferably greater than 30 microns, more preferably greater than 40 microns. The powder of the oat protein composition presents advantageously a mean particle size lower than 300 microns, preferably lower than 200 microns, more preferably lower than 150 microns. The powder is preferentially a spray-dried powder.

In the present application “particle size” must be understood as a notion introduced for comparing dimensions of solid, liquid or gaseous particles. The particle-size distribution (PSD) of a powder, or granular material, or particles dispersed in fluid, is a list of values or a mathematical function that defines the relative amount, typically by mass, of particles present according to size. Several methods can be used for measuring particle size and particle size distribution. Some of them are based on light, or on ultrasound, or electric field, or gravity, or centrifugation. The use of sieves is a common measurement technique. In the present application, the use of laser diffraction method is preferred. As for “mean particle size” (d 50) determined by laser diffraction, this mean particle size is a volume-weighted mean particle size. The man skilled in the art will be able to select a laser diffraction method allowing him to obtain an accurate mean particle size determination. For example, d 50 can be measured by a laser granulometry apparatus (Mastersizer 3000, from Malvern), which measures intensity of scattered light across a range of scattering angles using forward scattering measurement, on a dry powder without dispersion buffer, and using the software of the apparatus with the Mie scattering model to fit the distribution to the measured scattering pattern.

The oat protein composition of the invention can also present an improved color compared to existing commercial oat protein concentrates, especially when using a step of ultrasonication treatment. The powder can have parameters a* between −4 to 4, for example between −2 and 2, preferably between −1 and 1 and L* above 75, preferably above 80 when measured in the CIELAB color space. The parameter b* can be lower than 30, advantageously lower than 20, preferably lower than 15, by example lower than 10. L*, a* and b* are parameters known in the art and can be determined using classic color CIELAB color space, for example it can be characterized using a CR-5 device from Konica Minolta following the instructions manual.

In a preferred embodiment the oat protein composition presents improved acid-gelling properties. According to the invention, acid-gelling properties can include storage modulus as determined when using a TEST A.

In a preferred embodiment the oat protein composition presents improved thermal gelling properties. According to the invention, thermal-gelling properties can include gain in storage modulus as determined when using a TEST B.

The Applicants have shown that the oat protein composition according to the present invention, particularly the oat protein composition obtained by the process comprising a heat treatment step as described below, displays improved gelling properties.

One of the advantages of the invention is that the protein composition can be an acid-gelling oat protein composition that has high gelling properties when put at acidic pH. By “acid-gelling protein composition”, it is meant a protein composition having a storage modulus (acid G′) of at least 200 Pa when determined using a TEST A.

The acid-gelling oat protein composition can have a storage modulus of at least 250 Pa when determined using a TEST A, advantageously at least 260 Pa, or at least 270 Pa, or at least 280 Pa, or at least 290 Pa, or at least 300 Pa, or at least 310 Pa, or at least 320 Pa, or at least 330 Pa, or at least 340 Pa, or at least 350 Pa, or at least 360 Pa, or at least 370 Pa, or at least 380 Pa, or at least 390 Pa, or at least 400 Pa, or at least 410 Pa, or at least 420 Pa.

Details of how to carry out the TEST A and determine the acid storage modulus (acid G′) can be found in the example section.

Another advantage of the invention is that the protein composition can be an thermal-gelling oat protein composition that has high gelling properties when heated at 80° C. By “thermal-gelling protein composition”, it is meant a protein composition having a gain in the storage modulus (thermal ΔG′) of at least 500 Pa when determined using a TEST B.

The thermal-gelling oat protein composition can have a gain in the storage modulus of at least 600 Pa when determined using a TEST B, advantageously at least 700 Pa, or at least 800 Pa, or at least 850 Pa, or at least 900 Pa, or at least 950 Pa, or at least 1000 Pa, or at least 1050 Pa, or at least 1100 Pa.

Details of how to carry out the TEST B and determine the gain in the storage modulus (thermal ΔG′) can be found in the example section.

The powder composition generally comprises, based on the total weight of the composition, a water content lower than 10%, generally between 3 and 7%.

Another object of the invention concerns a process of manufacturing an oat protein composition comprising:
a step of providing a suspension of an oat proteic product, the oat proteic product having a protein content higher than 50% and a molecular weight Mw above 30000 g/mol,
a treatment step of this oat proteic product suspension, the treatment being chosen from an ultrasonication treatment step and an heat treatment step wherein, in the case of the heat treatment of the suspension:
   the pH of the suspension is between 8 and 11,
   the temperature of the suspension is between 120 and 180° C., wherein no enzymatic treatment step using endoprotease is done after the treatment step of the oat proteic product suspension.

This process is able to improve the solubility of the oat proteic product provided. This process allows the production of the oat protein composition of the invention defined above.

The suspension of an oat proteic product can simply be provided by blending oat proteic product with a liquid, preferably water. As water, any food compatible water can be used, but tap water, reverse osmosis water and deionized water are preferred.

This oat proteic product has a protein content higher than 50% by weight of protein or above, based on dry matter based on the total dry weight of the oat proteic product. The oat protein proteic can have a protein content chosen to achieve the desired protein content of the oat protein composition. It can be for example from around 55 to 90% by weight of protein based on dry matter based on the total dry weight of the oat proteic product. In a general manner, the oat proteic product can present similar protein contents to what is indicated in the description of the oat protein composition section.

This oat proteic product has a molecular weight Mw above 30000 g/mol. The oat protein proteic can have a molecular weight Mw chosen to achieve the desired protein content of the oat protein composition. Indeed, one advantage of this process is that it allows the improvement of the solubility without strongly affecting the molecular weight of the protein. In a general manner, the oat proteic product can present similar molecular weight Mw to the molecular weight Mw of the oat protein composition obtained, i.e. a molecular weight Mw lower than 110% of the molecular weight Mw of the oat protein composition obtained, generally between 95 and 105%.

Advantageously, the oat proteic product has a deamidation degree of the protein below 40%, preferably below 20%, for example below 15%, or below 10%, or below 5% or around 0%. Indeed, one advantage of this process is that it allows the improvement of the solubility without strongly affecting the deamidation degree of the protein. In a general manner, in the process of the invention, the oat protein composition obtained can present similar deamidation degree to the deamidation degree of the oat proteic product, i.e. a deamidation degree not higher than the deamidation degree of the oat proteic product plus 10%, preferably not higher than the deamidation degree of the oat proteic product plus 5%. More preferably, the oat proteic product has approximatively the same deamidation degree than the oat protein composition obtained.

Advantageously, the oat proteic product is not substantially chemically substituted.

In an embodiment, the solubility of the oat proteic product at pH 7 is below 40%, for example below 30% or below 20%.

In a general manner, the oat proteic product can have similar contents to the oat protein composition defined above in terms of lipid, starch, starch hydrolysate, soluble fiber and insoluble fiber.

In an embodiment, the oat protein composition has an extractable lipid content below 10% by weight on dry matter based on the total dry weight of the oat protein composition. To obtain that composition, the oat proteic product may be obtained by a process comprising a delipidation step. However, it is preferable that the oat protein composition does not comprise traces of organic solvent, i.e. contains less than 100 ppm of solvent. Thus, it is preferable that the oat proteic product does not comprise traces of organic solvent either. Inventors have developed different processes that allow to obtain such oat proteic products that do not comprise traces of organic solvent detailed below.

A first process to obtain such oat proteic product is described in the unpublished patent application PCT/EP2020/068658. In this document, the oat proteic product does not contain traces of organic solvent, has residual lipid content below 10% by weight on dry matter based on the total dry weight of the oat protein composition and has a mean particle size (d 50), determined by laser diffraction, greater than 10 microns. This document also discloses a process for producing an oat proteic product which has a residual lipid content below 10% by weight on dry matter based on the total dry weight of the oat proteic product characterized in that the process comprises the following steps:
1) Preparing oat seeds or provide a protein rich flour;
2) In the case of using oat seeds in step 1, grinding oat seeds of step 1 until obtaining a protein rich flour;
3) Mixing the protein rich flour of step 1 or 2 with water until obtaining a protein rich suspension;
4) Adjunction of an amylase enzyme to the protein rich suspension of step 3 thereby hydrolyzing the protein rich suspension;
5) Optionally separating by centrifugation the hydrolyzed protein rich suspension of step 4 until obtaining a heavy layer comprising fibers and a light layer comprising proteins;
6) Adjunction of polysorbate to the hydrolyzed protein rich suspension of step 4 or optionally to the light layer comprising proteins of step 5;
7) Separating by centrifugation the protein rich suspension comprising polysorbate or light layer comprising proteins of step 6 in an heavy layer containing proteins and a light layer containing soluble compounds including lipids; and
8) Optionally drying the heavy layer containing proteins of step 7.

A second process to obtain such oat proteic product is described in the unpublished patent application EP21305003. The document describes a powder of an oat proteic product characterized in that said product does not contain any traces of organic solvent, has protein content higher than 55%, has a extractable lipid content below 10% by weight on dry matter based on the total dry weight of the oat protein composition, a starch: soluble fiber mass ratio above 1 and has a mean particle size d-50 greater than 10 microns. This process for producing an oat proteic product which has a extractable lipid content below 10% by weight on dry matter based on the total dry weight of the oat proteic product is characterized in that the process comprises the following steps:
preparing a protein-rich suspension from oat starting material;
separating a soluble fraction comprising protein from an insoluble fraction comprising starch and fibers;
A step of adding a starch hydrolysate and a polysorbate to the soluble fraction and optionally adjusting pH from 5.8 to 8.0, preferably 6-7 to form additive-containing soluble fraction;
A step of heating the additive-containing soluble fraction at a temperature going from 35 to 80° C.;
A step of forming a proteic precipitate;
A step of separation of the proteic precipitate from solubles components to obtain a protein curd
Optionally at least one washing step of the protein curd, preferably at a pH between 4.5 and 6 and at a temperature between 50 and 60° C.
Optionally a step of adjusting the pH of the protein curd at a range going from 6.5 to 10.
Optionally a step of heat treatment of the protein curd
Optionally a step of homogenization treatment

The contents of all of these patent applications PCT/EP2020/068658, and EP21305003 are incorporated by reference. The expression “oat protein composition” as used in these patent applications corresponds to the “oat proteic product” which is used as starting material in the present application.

The suspension of the oat proteic product can generally comprise from 2 to 20% oat proteic product in the suspension. Even if this suspension may comprise other optional components than the oat proteic product, such as salts, these optional components are generally present in small quantities. Preferably the suspension comprises less than 0.5% of NaCl, for example less than 0.25% of NaCl. Preferably the suspension comprises less than 0.5% of added salts, for example less than 0.25% of added salts. The rest of the suspension consists in solvent and is generally water.

In the embodiment where the treatment comprises a heat treatment of the suspension:
the pH of the suspension is between 8 and 11,
the temperature of the suspension is between 120 and 180° C.

Advantageously, the pH of the suspension is from 8.2 to 10.5, preferably from 9.0 to 10.0. The temperature of the suspension during heat treatment is advantageously between 130 and 170° C., preferably between 135 to 165° C., more preferably between 145 and 160° C. This step has preferably a duration of 1 to 600 s, advantageously of 2 to 60 s, more advantageously of 5 to 30 s, for example between 10 and 20 s. The suspension may be heated by direct heating systems such as steam injection or infusion-based or by indirect heating systems or heat exchangers such as plate exchanger, tubular exchanger or scraped-surface exchanger. Preferably, this step is done by direct steam injection. The inventors have observed that the solubility tends to increase with a higher pH and a higher temperature but time of the heat treatment has less impact. One can refer to the Example 2. As the composition and properties of the oat proteic product has an impact on the final properties of the oat protein composition of the invention, the conditions can thus be adapted by the man skilled in the art in order to reach the targeted solubility. In a general manner, when the pH is from 8 to 8.5, the conditions of heat treatment can be a temperature going from 140 to 180° C. for the time ranges indicated above, preferably from 140 to 155° C. Alternatively, when the pH is from 8.5 to 11 or from 9 to 10, the conditions of heat treatment can be a temperature going from 120 to 180° C. for the time ranges indicated above, preferably from 130 to 155° C. or from 138 to 155° C.

Preferably, in this embodiment, the process comprises a step of cooling that takes place after the heat treatment, preferably a step of flash cooling.

Alternatively, the treatment step may be an ultrasonication step.

In this embodiment, the suspension can have a pH going suspension has a pH from 4 to 11, advantageously from 6 to 11, preferably from 7 to 10, more preferably from 7 to 8.5 during the ultrasonication step.

To set the pH during the process of the invention, any inorganic or organic base or acid reactant can be used. It may be chosen from caustic soda, potash, lime, citric acid, ascorbic acid, nitric acid, sulfuric acid and hydrochloric acid.

When the process comprises an ultrasonication step, advantageously the suspension has temperature from 15 to 90° C., advantageously from 20 to 70° C., generally 20 to 50° C. during the ultrasonication step.

Advantageously, the total energy input delivered per unit gram of oat proteic product is from 5 to 200 kJ/g, preferably from 7 to 100 kJ/g, for example from 10 to 80 kJ/g.

In the invention, the total energy input delivered by the ultrasonic device to the liquid material can be calculated and expressed as the total power (W) applied with the sonication time (s) per g of oat proteic product (J/g). The quantity of oat proteic product is directly related to the total weight and the dry matter of the suspension.

This step can be done using all kinds of ultrasonicators, for example ultrasonic baths, probe sonicators or ultrasonic processors.

It is possible to apply a batch process or a continuous process depending on the configuration of the ultrasonic device used.

As possible ultrasonic devices that can be achieve the process of the invention, QSonic Q 500 Ultrasonic Processor can be cited. For example, when using a Q500 processor that holds 500 watts (25 KHz Frequency, 1000 VRMS output voltage) and applying this power during 10 minutes to 200 g of solution having a dry matter of 5%, it follows that the total energy input delivered per unit gram of suspension oat proteic product will be:

500 W×10×60 s/(200 g× 5/100)=30 kJ/g.

The man skilled in the art is able to scale up the process by knowing the quantity of oat proteic product to be treated. He can adapt treatment time, power and size of the device to be purchased and/or dry matter to be treated.

The process of the invention does not comprise any enzymatic treatment step using endoprotease after the treatment step of the oat protein suspension. Endoproteases are enzymes which can reduce significantly the weight average molecular weight of large amino acids because the hydrolysis takes place inside the amino acids chains.

Enzymes are generally classified using the Enzyme Commission number (EC number), which is a numerical classification scheme for enzymes, based on the chemical reactions they catalyze. Main classes of enzymes able to hydrolyze proteins are protease and hydrolases acting in linear amides on carbon-nitrogen bonds other than peptide bonds. Proteases are classified according to this classification under the EC number 3.4. Hydrolases acting in linear amides on carbon-nitrogen bonds other than peptide bonds are classified according to this classification under the EC number 3.5.1. These hydrolase enzymes EC 3.5.1 encompass for example glutaminase.

In an embodiment, the process of the invention comprises an enzymatic step using an enzyme able to hydrolyze proteins different from endoprotease, advantageously using hydrolases of EC 3.5.1, for example using glutaminase. In a preferred mode of this embodiment, the enzymatic step is applied on the oat proteic suspension before the step of treatment of the suspension.

However, it is not needed to use enzymes in the process of the invention. In an embodiment, the process of the invention does not comprise any enzymatic treatment step using protease after the treatment step of the oat protein suspension. In an embodiment, the process of the invention does not comprise any enzymatic treatment step using glutaminase after the treatment step of the oat protein suspension. In an embodiment, the process of the invention does not comprise any enzymatic treatment step using hydrolases of EC 3.5.1 after the treatment step of the oat protein suspension. In an embodiment, the process does not comprise any enzymatic treatment step using endoprotease. In an embodiment, the process of the invention does not comprise any enzymatic treatment step using protease. In an embodiment, the process of the invention does not comprise any enzymatic treatment step using glutaminase. In an embodiment, the process of the invention does not comprise any enzymatic treatment step using hydrolases of EC 3.5.1. Each of the embodiments regarding the use of enzymes described in this paragraph can of course be combined. To obtain the characteristics of the oat protein composition defined above, it is preferable that the process does not comprise any enzymatic treatment step that catalyzes protein hydrolysis to maintain both deamidation degree and molecular weight of the protein.

The process can comprise a combination of these heat treatment and/or ultrasonication steps. After these treatment steps, an oat protein composition having improved solubility is obtained. Generally, the solubility of the oat protein composition is higher than the sum of the solubility of oat proteic product and 10%, advantageously higher than the sum of the solubility of oat proteic product and 20%, more advantageously higher than the sum of the solubility of oat proteic product and 30%, even more advantageously higher than the sum of the solubility of oat proteic product and 40%, preferably higher than the sum of the solubility of oat proteic product and 50%, more preferably higher than the sum of the solubility of oat proteic product and 60%, even more preferably higher than the sum of the solubility of oat proteic product and 70%.

Advantageously, the process further comprises a step of adjustment of the pH of the oat protein composition to a pH of between 6.0 to 8.0, preferably around 7.0.

Preferably, the process further comprises an homogenization step that can advantageously take place just after the treatment step(s) of the suspension, eventually after adjustment of the pH. Homogenization techniques in the field of proteins are known in the art. In particular, the homogenization may be carried out at high pressure. For example, it can be at a pressure of between 2 MPa and 800 MPa, for example between 2 MPa and 250 MPa.

The process can also comprise an additional step of heat treatment of the oat protein composition. The temperature can be from 75 to 180° C. Depending on the temperature, the time of the treatment can be from 0.1 s to 20 minutes, advantageously from 0.1 to 30 s, most preferably between 10 and 20 s.

The process can further comprises a step of drying the oat protein composition, preferably a spray drying step. This step allows to obtain the oat protein composition in the form of a powder. The drying step can be done by using drum drying or spray drying, including multi-stage spray drying, preferably spray drying. It is also possible to use different particle size selection steps, such steps of grinding the dried material and/or of sieving and/or using zig zag separators to obtain a selected particle size.

A third and last embodiment of the present invention is the use of the oat protein composition of the present invention or obtained by the process of the present invention, preferably in food, feed, pharmaceutical and cosmetic fields. More particularly, the invention concerns a food or beverage comprising the oat protein composition of the invention.

Such oat protein composition is particularly suitable for ready to drink beverages, non-dairy beverages or powder mixes. Its high solubility and high molecular mass allows an improved organoleptic experience when formulated.

Such oat protein composition is particularly suitable for ready to drink beverages or baking or any other food application such as protein bars, non-dairy beverages, powder mixes, yogurts, cheeses, or meat-like products. Its low lipid content allows an improved organoleptic experience when formulated, Indeed, when a product comprises high amounts of lipids, these undesirable lipids can get oxidized and develop a rancid taste, which negatively affects the organoleptic quality of the product.

In general terms, the oat protein composition of the invention can be used in food and beverage products that may include the oat protein composition in an amount of up to 100% by weight relative to the total dry weight of the food or beverage product, for example in an amount of from around 1% by weight to around 80% by weight relative to the total dry weight of the food or beverage product. All intermediate amounts (i.e. 2%, 3%, 4% . . . 77%, 78%, 79% by weight relative to the total weight of the food or beverage product) are contemplated, as are all intermediate ranges based on these amounts.

Beverages include acid beverages, carbonated beverages (including, but not limited to, soft carbonated beverages); non-carbonated beverages (including, but not limited to, soft non-carbonated beverages such as flavored waters, fruit juice and sweet tea or coffee based beverages); beverage concentrates (including, but not limited to, liquid concentrates and syrups as well as non-liquid ‘concentrates’, such as freeze-dried and/or powder preparations). The protein content in the beverage can be very different and the beverage can be a high protein drink. The content is for example between 1 and 12% of the total mass of the beverage, for example between 2 and 10%. Beverages also include milk-like beverages, that can be «barista» type or «coffee creamer» type.

Food products which may be contemplated in the context of the present invention include baked goods; sweet bakery products (including, but not limited to, rolls, cakes, pies, pastries, and cookies); pre-made sweet bakery mixes for preparing sweet bakery products; pie fillings and other sweet fillings (including, but not limited to, fruit pie fillings and nut pie fillings such as pecan pie filling, as well as fillings for cookies, cakes, pastries, waffles, pancakes, muffins and biscuits, confectionary products and the like, such as fat-based cream fillings); desserts such as flan, custard, gelatins and puddings; frozen desserts (including, but not limited to, frozen dairy desserts such as ice cream—including regular ice cream, soft serve ice cream and all other types of ice cream—and frozen non-dairy desserts such as non-dairy ice cream, sorbet and the like); snack bars (including, but not limited to, cereal, nut, seed and/or fruit bars); bread products (including, but not limited to, leavened and unleavened breads, yeasted and unyeasted breads such as soda breads, breads comprising any type of wheat flour, breads comprising any type of non-wheat flour (such as oat, potato, rice and rye flours), gluten-free breads); pre-made bread mixes for preparing bread products; sauces, syrups and dressings; sweet spreads (including, but not limited to, jellies, jams, butters, nut spreads, dulce de leche and other spreadable preserves, conserves and the like); confectionary products (including, but not limited to, jelly candies, soft candies, hard candies, chocolates, caramels and gums); sweetened and un sweetened breakfast cereals (including, but not limited to extruded breakfast cereals, flaked breakfast cereals and puffed breakfast cereals); and cereal coating compositions for use in preparing sweetened breakfast cereals. Other types of food and beverage product may also be contemplated in the context of the present invention. In particular, animal foods (such as pet foods) are explicitly contemplated.

Oat protein can be used in combination with flavours or masking agents.

Oat protein can also be used, eventually after texturization, in meat-like products such as emulsified sausages or plant-based burgers, fish-like products or seafood-like products. It can also be used for making egg substitutes or for the manufacturing of protein containing products such as tofu or tempeh. «Texturized proteins» generally means proteins texturized by extrusion, i.e. especially by dry extrusion to make Textured Vegetable Protein, wet extrusion or high moisture extrusion. Extruders can be single screw extruders, twin screw extruders, multiple screw extruders. Example of multiple screw extruders are planetary extruder or ring-extruder. Other technologies such as shear cell technology, microextrusion or 3D printing can also be used.

The food or beverage product can be used in specialized nutrition, for specific populations, for example for baby or infants, teenagers, adults, elderly people, athletes, people suffering from a disease. It can be meal substitutes formulations, complete nutrition beverages, for example for weight management or in clinical nutrition (for example tube feeding or enteral nutrition).

The oat protein composition can be used as the sole source of protein but also can be used in combination with other plant or animal proteins. These other proteins can be hydrolyzed or not. Generally, these are in the form of isolates or concentrates. The term “plant protein” denotes all the proteins derived from cereals, oleaginous plants, leguminous plants and tuberous plants, and also all the proteins derived from algae and microalgae or fungi, used alone or as a mixture, chosen from the same family or from different families. In the present application, the term “cereals” is intended to mean cultivated plants of the grass family producing edible grains, for instance wheat, rye, barley, maize, sorghum or rice. The cereals are often milled in the form of flour, but are also provided in the form of grains and sometimes in whole-plant form (fodders). In the present application, the term “tubers” is intended to mean all the storage organs, which are generally underground, which ensure the survival of the plants during the winter season and often their multiplication via the vegetative process. These organs are bulbous owing to the accumulation of storage substances. The organs transformed into tubers can be the root e.g. carrot, parsnip, cassava, konjac), the rhizome (e.g. potato, Jerusalem artichoke, Japanese artichoke, sweet potato), the base of the stalk (more specifically the hypocotyl, e.g. kohlrabi, celeriac), the root and hypocotyl combination (e.g. beetroot, radish). For the purposes of the present invention, the term “leguminous plants” is intended to mean any plants belonging to the family Cesalpiniaceae, the family Mimosaceae or the family Papilionaceae, and in particular any plants belonging to the family Papilionaceae, for instance pea, bean, soy, broad bean, horse bean, lentil, alfalfa, clover or lupin. This definition includes in particular all the plants described in any of the tables contained in the article by R. Hoover et al., 1991 (Hoover R. (1991) “Composition, structure, functionality and chemical modification of legume starches: a review” Can. J. Physiol. Pharmacol., 69, pp. 79-92). Oleaginous plants are generally seed-producing plants from which oil is extracted. Oilseed plants can be selected from sunflower, rapeseed, peanut, sesame, pumpkin or flax. The animal protein can be for example egg or milk proteins, such as whey proteins, casein proteins or caseinate. The oat protein composition can thus be used in combination with one or more of these proteins or amino acids in order to improve the nutritional properties of the final product, for example to improve the PDCAAS of the protein or to bring other or modify functionalities.

The oat protein can also be used for the manufacturing of pharmaceutical products or in fermentation, for example for the production of fungi metabolites or cell culture metabolites.

The oat protein composition of the invention can also be used for acidic food products such as yogurts (including, but not limited to, full fat, reduced fat and fat-free dairy yogurts, as well non-dairy and lactose-free yogurts and frozen equivalents of all of these), cheeses or acidic sauces. Acidic food products can have a pH of 3 to 6 when diluted at a dry matter of 10%. The oat protein composition can be used to form of a milk and fermented and/or acidified to provide yogurts and cheeses. These milks can present a dry matter going from 5 to 30%. These milks can comprise other components such as sugars and fats and optional, Yogurts can include stirred yogurts, set yogurts or yogurts to drink. These can be flavoured or not and can include other components such as fruit preparations and/or sweeteners. Cheeses can be process cheese, swiss cheese, string cheese, ricotta, provolone, parmesan, muenster, mozzarella, jack, manchego, blue, fontina, feta, edam, double Gloucester, cheddar, asiago and Havarti. Acidic sauces are for example mayonnaise or ketchup.

The invention will be better understood with the following non-exhaustive examples.

### EXAMPLES

Methods

Determination of Solubilities:

Powder sample method: Pre-heat the oven to 130° C. Get the weight of the 100 mL beaker and small stir bar, then add 1.25 g of the protein powder to it, all while using the analytical balance. Record the total weight of beaker, stir bar and protein powder. Add about 35.00 g of de-ionized water (RT, 20±2° C.) and stir sample until the sample is completely dissolved. The stir plate is usually set at around 400 rpm. Adjust the pH of the solution with either 1N HCl or NaOH to reach the targeted pH (4 or 7 in the following examples). Once pH is adjusted, add enough water to bring the total volume up to 50 g, then record the total weight of the sample, beaker, and water. Stir the sample for 30 minutes at around 700 rpm. Transfer the sample to a 50 mL centrifuge tube. Centrifuge the sample at 3.000×g for 15 minutes. Transfer the supernatant to a beaker and stir the sample to insure the supernatant is homogenized. Get the weight of a small aluminum pan and add about 15 g of the supernatant. Record the total weight. Place the samples in the oven for 75 minutes or until completely dry. Once dried, place the samples in the desiccator for 30 minutes to cool off and record the weight of the pan and dried sample.

Liquid sample method: Pre-heat the oven to 130° C. Get the solid content of the liquid sample and dilute the sample to 2.50% solid (with deionized water) and a total sample weight of 60 mL. This would be done for every desirable pH. Adjust to the desired pH with either 1N HCl or NaOH to reach the targeted pH (4 or 7 in the following examples). Stir the sample for 30 minutes at around 700 rpm on the stir plate. Take the weight of two small aluminum pans on the analytical scale. After 30 minutes of stirring, transfer about 40 mL of the sample into a 50 mL centrifuge tube, transfer the rest to one of the aluminum dishes. Centrifuge the sample at 3.000×g for 15 minutes. Transfer the supernatant to a beaker and stir the sample to insure the supernatant is homogenized. Add about 15 g of the supernatant to the other pan. Record the total weight. Place both pans in the oven for 75 minutes or until the sample is completely dried. Once dried, place the samples in the desiccator for 30 minutes to cool off and record the weight of the pan and dried sample.

% protein: Protein content is % N6.25 and nitrogen content is determined using combustion analyzer-Elementer, with AOAC 997.09 method.

% lipids: extractable lipid content is determined using Soxhlet extraction in petroleum ether using AOAC 963.15 method.

Explanations on how to measure the molecular weight and deamidation degree can be found above in the description of the patent application.

### Example 1: Manufacturing of Freeze Dried Powder of Soluble Oat Protein Isolate Using Heat Treatment

The following process was done to manufacture the proteic product: Weigh 12.5 kg oat flour (Richardson Milling low fiber), fill a jacketed tank of around 190 L with approximately 88 L of 50° C. water, mix the flour into water and adjust to achieve 10.5% solids. Adjust the pH to 5.5 with HCl while agitating for 10 min and add 125 g Liquozyme supra (from Novozyme). Heat to 70° C. with heat exchanger blended with a recirculating pump during 2 hours. Add 420 g of 30% solution of polysorbate Tween 80 (equivalent to 125 g of dry polysorbate). Heat to 65° C. and hold 60 min, with a recirculating pump. Fill tank to capacity with water, heat to 60° C. Reduce pH to 5.0 with HCl. After precipitation, centrifuge (Clara 20, 0.45 m³/h, 9,000 rpm). Wash the proteic precipitate fraction in jacketed tank (around 190 L): fill to capacity with water, heat to 60° C. and adjust again pH to 5.0 with HCl. Centrifuge again and recover this oat proteic product suspension.

Divide this oat proteic product suspension into three, store overnight at 3° C.:
Sample 1-1—freeze dry as-is (control untreated)
Sample 1-2, adjust to pH 7.0 (reference)
Sample 1-3, adjust to pH 9.5 with 1N aqueous solution of caustic soda (invention)

On samples 2 and 3, apply during 30 s at around 154° C. using direct steam injection, and cool immediately the sample at 71° C. (flash cooling). The samples were then freeze-dried.

The oat protein isolate was analyzed: 73.0% of protein and 7.7% extractable lipid.

In the Table 1 are reported the solubility at pH 4 and pH 7 for the 3 samples, the deamidation degree and the molecular weight Mw and the molecular weight profile.

TABLE 1
solubility of samples 1-1 to 1-3

| Sample no | Sample 1-1 | Sample 1-2 | Sample 1-3 |
|---|---|---|---|
| Solubility at pH 7 | 10% | 28% | 81% |
| Solubility at pH 4 | 3% | 9% | 21% |
| Mw (g/mol) | 65110 | 52700 | 68210 |
| % MW >300000 g/mol | 7.72 | 4.31 | 14.31 |
| % MW 50000 to 300000 | 56.87 | 51.83 | 47.41 |
| g/mol | | | |
| % MW 10000 to 50000 g/mol | 34.04 | 35.65 | 30.51 |
| % MW <10000 g/mol | 1.37 | 8.21 | 7.77 |
| Deamidation degree | <1% | <1% | <1% |

The results above demonstrate that no deamidation nor hydrolysis of the protein occurred during the process of the invention. The solubility of the oat protein sample is strongly increased at pH 7 and, to a smaller extent, at pH 4. Oat globulins have an isoelectric point which is around 5 and are not soluble at this pH. The fact that, at a pH of 4, the solubility of the oat protein composition is still low is another proof that the globulins, which are the main protein of the composition contained in the composition, are not strongly hydrolyzed, deamidated or chemically substituted. Inventors have demonstrated that, even low, the solubility at pH 4 of the oat protein composition is 7 times higher that when untreated which can improve its behavior at acid pH in some applications, for example in acid beverages.

### Example 2: Study of the Conditions of the Heat Treatment

In this example conditions of pH, time and temperature of the heat treatment were studied.

The process was the following: Fill a 190 L jacketed tank with approximately 160 L of 50° C. water. Mix 50 lb (22.7 kg) of flour into water, adjust to achieve 10.5% of dry matter. Adjust pH to 5.4 to 5.5 with HCl while agitating for 10 min then add 230 g Liquozyme supra (from Novozyme). Heat to 70° C. with heat exchanger, while using a recirculating pump during 2 hours. Adjust the pH to 7.0 with NaOH. Centrifuge feed to Lemitec (5000 rpm, 10 rpm diff, 1500 ml/min, with 60/10 weir), and collect overflow in 380 L jacketed tank. Add 115 g of solution of polysorbate (30% dry solids in water) to the tank. Heat to 65° C., adjust pH to 6.5 and hold 60 min, while recirculating with centrifugal pump. Fill the tank to capacity with water, heat back to 60° C. and reduce the pH to 5.0 with HCl. Centrifuge on Clara 20 (0.45 m³/h, 9000 rpm) and take underflow. Resuspend the underflow fraction in jacketed 380 L tank, fill to capacity with water, heat to 60° C. and adjust pH to 5.0 with HCl. Centrifuge on Clara 20 (0.45 m³/h, 9000 rpm) and take the underflow fraction. Store this oat proteic product suspension overnight in fridge and heat treat following using direct steam injection in the conditions in Table 2 below, then cooled by flash cooling a 71° C. Freeze dry the samples. The composition was analyzed: 79.4% of protein, 8.5% of extractable lipids.

TABLE 2
conditions of heat treatment and solubility

| pH | Time (s) | Temp (° C.) | Solubility at pH 7 (%) |
|---|---|---|---|
| 7 | 20 | 132 | 31 |
| 7 | 10 | 141 | 32 |
| 7 | 30 | 141 | 39 |
| 7 | 20 | 149 | 40 |
| 8.25 | 10 | 132 | 46 |
| 8.25 | 30 | 132 | 44 |
| 8.25 | 20 | 141 | 52 |
| 8.25 | 10 | 149 | 61 |
| 8.25 | 30 | 149 | 52 |
| 9.5 | 20 | 132 | 70 |
| 9.5 | 10 | 141 | 78 |
| 9.5 | 30 | 141 | 82 |
| 9.5 | 20 | 149 | 92 |

| | |
|---|---|
| No treatment | 25 |

To achieve high solubility, the most important parameters in the process are temperature and pH conditions.

### Example 3: Manufacturing of Spray Dried Powder of Soluble Oat Protein Isolate Using Heat Treatment

The process was the following: Fill a 190 L jacketed tank with approximately 160 L of 50° C. water. Mix 28 kg of flour (Richardson Milling low fiber raw material) into water, adjust to achieve 10.5% of dry matter. Adjust pH to 5.4 to 5.5 with HCl while agitating for 10 min then add 280 g Liquozyme supra (from Novozyme). Heat to 70° C. with heat exchanger, while using a recirculating pump during 2 hours. Adjust the pH to 7.0 with NaOH. Centrifuge feed to Lemitec (5000 rpm, 10 rpm diff, 2000 ml/min, with 60/10 weir), and collect overflow in 380 L jacketed tank. Add 115 g of solution of polysorbate (30% dry solids in water) to the tank. Heat to 65° C., adjust pH to 6.5 and hold 60 min, while recirculating with centrifugal pump. Fill the tank to capacity with water, heat back to 60° C. and reduce the pH to 5.0 with HCl. Centrifuge on Clara 20 (0.45 m³/h, 9000 rpm) and take underflow. Resuspend the underflow fraction in jacketed 380 L tank, fill to capacity with water, heat to 60° C. and adjust pH to 5.0 with HCl. Centrifuge on Clara 20 (0.45 m³/h, 9000 rpm) and take the underflow fraction. Store this underflow fraction overnight in fridge at 3° C. This oat proteic product suspension was divided into three samples:
Sample 3-1—freeze dry as-is the oat proteic product suspension (control untreated)
Sample 3-2, adjust to pH 7.0, oat proteic product suspension passed through UHT at 154° C. using direct steam injection during 30 seconds, then flash cooled at 71° C. and freeze dried (reference)

The other portion (sample 3-3) of this oat proteic product suspension was adjusted to pH to 9.5 and passed through UHT at 154° C. using direct steam injection during 30 seconds, then flashed cool at 71° C. and spray-dried (220° C. inlet, 90° C. outlet). The composition was analyzed: 79.8% of protein, 4.5% of extractable lipids.

In the Table 3 are reported the solubility at pH 4 and pH 7 and the deamidation degree for the 3 samples.

TABLE 3
Solubility and deamidation degree of samples 3-1 to 3-3

| Sample no | Sample 3-1 | Sample 3-2 | Sample 3-3 |
|---|---|---|---|
| Solubility at pH 7 | 15% | 35% | 76% |
| Solubility at pH 4 | 4% | 19% | 19% |
| Deamidation degree | <1% | <1% | <1% |

The results above demonstrate again that no deamidation nor hydrolysis of the protein occurred during the process of the invention and this confirms that same effects are observed when using spray drying instead freeze drying.

In addition, the average molecular weight and the distribution of molecular weights of the proteins was measured, as described above in Example 1.

Sample 3-3 has an average molecular weight of 59431 g/mol and displayed the following distribution:
1.42% of proteins having a molecular weight of 300000 g/mol and more
26.19% of proteins having a molecular weight of between 50000 g/mol and 300000 g/mol,
36.12% of proteins having a molecular weight of between 10000 g/mol and 50000 g/mol
36.25% of proteins having a molecular weight of 10000 g/mol and less.

### Example 4: Heat Treatment of a Commercial Oat Protein Concentrate

A commercial oat protein concentrate (PROATEIN®) comprising 60% of protein, which is not chemically substituted nor deamidated, was used.

The process consists to weigh 150 g of the commercial oat protein as an oat proteic product. Add to 2500 g tap water, mix for 30 min at 25° C. in a mixer. Divide into three samples:
Sample 4-1—freeze dry as-is (control untreated)
Sample 4-2, adjust to pH 7.0 (reference)
Sample 4-3, adjust to pH 9.5 with 1N aqueous solution of caustic soda (invention)

On samples 4-2 and 4-3, apply during 30 s at around 154° C. using direct steam injection, and cool immediately the sample at 70° C. (flash cooling). Then the samples are homogenized on Pony homogenizer, 400 bar 1st stage, 40 bar 2nd stage. The samples were then freeze-dried.

In the Table 4 is reported the solubility at pH 7 for the 3 samples.

TABLE 4
Solubility of samples 4-1 to 4-3

| Sample no | Sample 4-1 | Sample 4-2 | Sample 4-3 |
|---|---|---|---|
| Solubility at pH 7 | 16% | 37% | 86% |

These results demonstrate that the process can be applied with success to commercial oat protein concentrates that have less protein content than the oat protein isolates manufactured in examples 1 to 3.

### Example 5: Manufacturing of Spray Dried Powder of Soluble Oat Protein Isolate Using Ultrasonication

The following process was used: Fill 190 L jacketed tank with approximately 174 L of water at 50° C. Mix 23 kg of flour into water, adjust to achieve 10.5% (+/−0.5%) solids. Adjust pH to 5.4 to 5.5 with HCl while agitating for 10 min. Add 230 g Liquozyme supra (from Novozyme). Heat to 70 C with heat exchanger, mixing with a recirculating pump during 1 hour. Adjust pH to 7.0 with NaOH. Centrifuge with Lemitec (5000 rpm, 10 rpm diff, 1500 ml/min feed, with 60/10 weir) and collect overflow in a 380 L jacketed tank. Add 115 g of solution polysorbate (aqueous solution at 30% of dry matter) to the tank. Heat to 65 C, adjust pH to 6.5, hold 60 min, recirculating with centrifugal pump. Fill in another 174 L of water to this tank with water, heat back to 60 C, Reduce pH to 5.0 with HCl. Centrifuge on Clara 20 (0.45 m³/h, 9,000 rpm) and take underflow fraction. Resuspend this fraction in jacketed 100 gal tank, fill in 320 L water, heat to 60° C. Adjust pH to 5.0 with HCl. Centrifuge again on Clara 20 (0.45 m³/h, 9,000 rpm). Store the oat proteic product in 19 L bucket overnight in fridge. Adjust pH to 7.5 while curd at 25° C. Treat by QSonic Q2000 Ultrasonic Processor for 10 or 20 minutes with 1 L per batch for this oat proteic product suspension at 10% DS. Repeat the process until all the suspension processed. Spray dry at 220° C. inlet, 90° C. outlet.

QSonic Q 2000 Ultrasonic Processor used with following characteristics;
a. 1.5 inch (38 mm) probe diameter, equals 11.34 cm2 cross section area,
b. 100 μm amplitude (vertical movement), 90% setting applied;
c. 2000 watt power, and 20 kHz frequency.

Using this process, the oat protein composition should present an approximate content of 80% of protein and an extractable lipid content well below 10%.

In this example, solubility was determined on the oat proteic suspension untreated, treated during 10 minutes and treated during 20 minutes. It was calculated that, with the ultrasonicator and the oat proteic product suspension at 10% of dry matter used, the ultrasonication treatment of 10 minutes delivers 12000 J/g of oat proteic product, whereas the ultrasonication treatment of 20 minutes delivers 24000 J/g of oat proteic product.

The results of solubility (liquid mode) are reported in Table 5

TABLE 5
Solubility of samples 5-1 to 5-3

| Sample no | Sample 5-1 | Sample 5-2 | Sample 5-3 |
|---|---|---|---|
| Solubility at pH 7 | 16% | 56% | 67% |

This trial demonstrates the ability to manufacture high solubility oat protein compositions thanks to ultrasonication at room temperature.

In addition, the average molecular weight and the distribution of molecular weights of the proteins was measured, as described above in Example 1.

Sample 5 according to the invention has an average molecular weight of 28681 g/mol and displayed the following distribution:
3.54% of proteins having a molecular weight of 300000 g/mol and more
11.32% of proteins having a molecular weight of between 50000 g/mol and 300000 g/mol,
52.83% of proteins having a molecular weight of between 10000 g/mol and 50000 g/mol
32.07% of proteins having a molecular weight of 10000 g/mol and less.

The deamidation degree of sample 5 was 0.02%.

### Example 6: Effect of Energy Provided and pH During Ultrasonication Step

For this example, the same oat proteic of example 5 was used. 3 samples were prepared with each of them, a dry substance was set to 5%, and the a adjusted 7, 7.5 and 8.5. The oat proteic product suspensions were treated using a QSonic Q500 Ultrasonic Processor for 10 or 20 minutes per batch (200 mL) for these oat proteic product suspension. QSonic Q 500 Ultrasonic Processor used with the following characteristics:
a. 0.5 inch (12.7 mm) probe diameter, equals 1.32 cm2 cross section area
b. 120 μm amplitude (vertical movement), 90% setting applied,
c. 500 watt power, and 20 kHz frequency.

The Table 6 reports solubility of different liquid samples tested and energy provided per g of protein.

TABLE 6
Solubility and energy provided

| Sample | pH | time | Solubility at pH 7 | Energy provided per g (J/g) |
|---|---|---|---|---|
| 6-1 | 7 | 0 | 14% | 0 |
| 6-2 | 7 | 10 | 61% | 30000 |
| 6-3 | 7 | 15 | 66% | 45000 |
| 6-4 | 7 | 20 | 67% | 60000 |
| 6-5 | 7.5 | 0 | 16% | 0 |
| 6-6 | 7.5 | 10 | 66% | 30000 |
| 6-7 | 7.5 | 15 | 69% | 45000 |
| 6-8 | 7.5 | 20 | 73% | 60000 |
| 6-9 | 8.5 | 0 | 18% | 0 |
| 6-10 | 8.5 | 10 | 71% | 30000 |
| 6-11 | 8.5 | 15 | 78% | 45000 |
| 6-12 | 8.5 | 20 | 82% | 60000 |

This example demonstrates that with success the possibility to obtain high solubility oat protein compositions using ultrasonication. It is observed that, with an energy above 30000 J/G, the solubility still increases but to a smaller extent. A higher pH during sonication allows to obtain higher solubility.

### Example 7: Ultrasonication Treatment of a Commercial Oat Protein Concentrate

A commercial oat protein concentrate (PROATEIN®) comprising 60% of protein, which is not chemically substituted nor deamidated, was used.

Prepare 329 g water in a stainless steel pot while stirring with overheat stirrer with 3-point agitator at 300 rpm. Add 21 g of oat protein concentrate into the water, 3 samples were prepared and, for each of them, adjusted to a pH of 7, 8.5 and 9.5. The oat proteic product suspensions were treated using a QSonic Q500 Ultrasonic Processor for 10 minutes per batch (200 mL, 6% dry matter) for these oat proteic product suspension. The treatment was done at 3 different temperatures: 25° C., 45° C. and 60° C.

The solubility (liquid mode) was reported for each of these conditions in Table 7.

TABLE 7
Solubility in function of pH and temperature of the suspension

| | | | |
|---|---|---|---|
| Sample | pH | Temperature | Solubility |

| | | |
|---|---|---|
| 7-1 (control) | No treatment | 12% |

| | | | |
|---|---|---|---|
| 7-2 | 7 | 25° C. | 58% |
| 7-3 | 8.5 | 25° C. | 67% |
| 7-4 | 9.5 | 25° C. | 83% |
| 7-5 | 7 | 45° C. | 59% |
| 7-6 | 8.5 | 45° C. | 75% |
| 7-7 | 9.5 | 45° C. | 87% |
| 7-8 | 7 | 60° C. | 58% |
| 7-9 | 8.5 | 60° C. | 67% |
| 7-10 | 9.5 | 60° C. | 90% |

These results demonstrate that the process can be applied with success to commercial oat protein concentrates that have less protein content than the oat protein isolates manufactured in examples 1 to 3. They also demonstrate that the temperature during ultrasonication is not strongly affecting the solubility of the oat protein composition obtained. On the opposite, the pH can have a significant impact.

### Example 8: Combination of a Glutaminase Treatment with Ultrasonication Treatment

Preparation of the OPI: The following protocol was followed: weigh 100 kg Grain Miller #70 oat flour, fill with 1000 L water (Tank size 2000 L) at 37° C. and mix flour gradually into water, make sure all flour dispersed well in water. Adjust the pH of the protein rich suspension to 2.0-2.1 with 2 N HCl while agitating and keep the temperature at 37° C., mix at 500 rpm for 90 minutes. Feed through Decanter (Centrisys) at 12 L/min, 3000 g-force, 3 rpm differential. The pellet (Heavy phase HP1) and supernatant (Light phase LP1) were collected. Take the LP1 fraction, heat the liquor up to 65° C., then add 3% (w/w) of maltodextrin (Glucidex®19) and add 0.8% (w/w) of polysorbate Tween 80, these amounts being based on the dry substance of the LP1 fraction. Adjust to pH 6.5 with 1 N NaOH, keep 66-68° C. and mix at 300 rpm for 60 min. Adjust pH to 5.2 and keep mix at 200 rpm for 15 min to form a protein precipitate. Feed through a disc-centrifuge at 7 L/min, 9800 rpm, discharge the solids every 90 sec. Collect protein curd (PPT1) and Overflow (O/F1). Dilute the protein curd (˜75 kg, target DS 12-14%) into 400 L of hot water (50° C.), mix at 200 rpm for 30 min. Adjust the pH to 5.2 and feed through a disc-centrifuge at 7 L/min, 9800 rpm, discharge the solids every 97 sec. Collect washed protein curd (PPT2) and Overflow (O/F2). Reconstitute the protein curd with water (10% DS), adjust pH to 7, then apply to a Jet cooker (130° C., 20-30 sec holding time). Submit to Spray drying with 180° C. inlet/90° C. outlet, at 12 L/hr feeding rate. Collect spray dried Oat Protein Isolate (OPI).

Different treatments were done using this OPI as oat proteic product to put into suspension and to be treated. A 6% solution OPI was prepared by mixing the OPI in water 50° C. and pH set at 7. Four treatments were conducted:
Sample 8-1: Incubate for 120 minutes at 50° C.,
Sample 8-2: Incubate in presence of protein glutaminase (PG-500) from Amano, at around 0.5% of the total weight of the suspension, for 120 minutes at 50° C.,
Sample 8-3: Ultrasonic treatment using QSonic Q500 Ultrasonic Processor (same settings than example 7) for 10 minutes at room temperature, sample cooling with water bath.
Sample 8-4: Apply the same ultrasonic treatment to a portion of sample 8-2

The solubility at pH 7 of the liquid samples obtained are reported in Table 8.

TABLE 8
Solubility of glutaminase and/or sonication treated proteins

| Sample | 8-1 | 8-2 | 8-3 | 8-4 |
|---|---|---|---|---|
| Solubility | 14% | 26% (+12%) | 52% (+38%) | 78% (+64%) |

This example demonstrates that the ultrasonication treatment allows to obtain a protein composition with high solubility. It also proves that the process of the invention can also improve the solubility of an oat protein that has been preliminary deamidated. These examples even show a synergistic effect: indeed if the effect was only cumulative, the solubility of the ultrasonicated deamidated oat protein would have a solubility increased by 50% (12+38), whereas this solubility is increased of 64%.

### Example 9: Gel Strength of the Oat Protein Compositions

Determination of the Storage Modulus Upon Acid Gelling (Test A)

To determine the storage modulus upon acid gelling (acid G′), 30 g of dry protein product (were dispersed at room temperature (around 22° C.) in 170 g of distilled water using a magnetic stirrer to produce a slurry having 15% of dry matter having a pH of 7. If the protein product is not neutral, a solution of HCl 1N or NaOH 1N partially replaces the water added in order that this slurry presents this pH of 7. A quantity of 0.02% sodium azide, expressed in dry weight, was added in the slurry to prevent bacterial growth. The slurries were stirred overnight to ensure complete hydration of the powders. After a duration of 12 hours, 2% glucono-delta-lactone (GDL) expressed in dry weight were added to the sample to slowly acidify the solutions to pH 4.6-4.8 over the course of several hours. The rheological properties (storage and loss moduli) were monitored maintaining the samples at 22° C. during acidification using a rheometer (Anton Parr Model MCR92) equipped with a concentric cylinder measuring system (CC39: cup diameter 42 mm; bob diameter 38.7 mm) that is filled with the recommended amount (approximately 65 g) applying a strain of 0.2% at a frequency of 1 Hz. The strain applied was within the linear viscoelastic region of the sample.

Acid Gel strength is expressed as the G′ storage modulus (Pa) after 300 min (5 hours of acidification) (acid G′).

Determination of the Storage Modulus Upon Thermal Gelling (Test B)

To determine the gain in storage modulus upon thermal gelling ((thermal ΔG′), 30 g of dry protein product (were dispersed at room temperature (around 22° C.) in 170 g of distilled water using a magnetic stirrer to produce a slurry having 15% of dry matter having a pH of 7. If the protein product is not neutral, a solution of HCl 1N or NaOH 1N partially replaces the water added in order that this slurry presents this pH of 7. A quantity of 0.02% sodium azide, expressed in dry weight, was added in the slurry to prevent bacterial growth. The slurries were stirred overnight to ensure complete hydration of the powders. After a duration of 12 hours, the storage modulus G′ is measured at 20° C. using a rheometer (Anton Parr Model MCR92) equipped with a concentric cylinder measuring system (CC39: cup diameter 42 mm; bob diameter 38.7 mm) that is filled with the recommended amount (approximately 65 g) applying a strain of 0.2% at a frequency of 1 Hz. The strain applied was within the linear viscoelastic region of the sample.

The sample is then heated and held at 80° C. for 2 hours, cooled back to 20° C. and the storage modulus after thermal treatment is measured again.

Thermal gel strength is measured as the difference between the G′ storage modulus (Pa) after 2 hours at 80° and the initial G′ storage modulus before heating (thermal ΔG′=G′ after heating−G′ before heating).

The compositions obtained in Examples 1, 3 and 5 were tested for gel strength according to Tests A and B. The results are presented in Table 9.

TABLE 9
Gel strength of gels obtained by acid gelling or thermal gelling

| Sample no | Sample 1 | Sample 3 | Sample 5 |
|---|---|---|---|
| Acid G′ | 316 | 418.8 | 66 |
| Thermal ΔG′ | 1090 | 1605 | 5 |

This example demonstrates that heat treatment according to the present invention allow to obtain a protein composition having superior gelling properties, such as acid gelling properties and thermal gelling properties.

### Example 10: Preparation of a Ready-to-Drink Beverage Comprising an Oat Protein Composition of the Invention

The following formula was prepared, as an alternative beverage to milk:

| Ingredient | % wt. |
|---|---|
| water | 92.19 |
| Oat protein powder | 3.74 |
| sugar | 2.80 |
| Gellan gum (Kelcogel HS-B) | 0.10 |
| Lecithin | 0.10 |
| Sunflower Oil | 1.17 |
| Total | 100 |

The quantity of ingredients were adjusted in order to obtain oat protein alternative beverage to milk having around 3.0 g of protein and around 1.5 g of fat per 100 mL of the finished milk prototype. These quantities are very close to the protein and fat contents of dairy milk.

The quantities indicated above were the ones used for the oat protein alternative beverage to milk of the invention.

The oat milk was prepared as follows:
1. Heat the oil and lecithin blend to 60° C. in a lab gravity convection oven
2. Hydrate the oat protein at 50° C. using Ross high shear mixer (HSM-100LC1-T) for 30 min (2500 rpm max)
3. Mix all the other dry ingredients with intense mixing using a whisk until achieve a complete dispersion
4. Change mixer stator from round hole stator to fine screen stator for high shear mixing
5. Set mixer stirring speed at 6000 rpm, ensure lecithin is well dispersed in oil
6. Add oil-lecithin blend into protein solution slowly. Mix for 5 min at 6000 rpm
7. Pour slurry through 150 micron screen into UHT
8. Start low agitation in feeding hopper and keep stirring at 300 rpm using VELP overhead mixer
9. Sterilize the solution by UHT indirect steam heat treatment (shell-in-tube): 142° C. for 5 sec
10. Homogenize (downstream) the solution at 75° C. using two-stage high-pressure homogenizer (GEA twin Panda 400) at 180 bars (1^{st} stage) and 20 bars (2^{nd} stage)
11. Bottle, seal and cool to 4° C.

The following oat protein compositions were used:
Example according to the invention: oat protein composition obtained by the process of Example 1
Comparative example 1: oat protein composition similar to the one of Example 7 of WO2021/001478
Comparative example 2: PROATEIN commercial oat protein concentrate

The following results were obtained:

The oat protein composition of the invention was easily processable compared to the comparative ones described in WO2021/001478 and PROATEIN: Indeed, clogging was observed when using the comparative oat proteins in this model recipe.

The advantage of the oat protein composition of the invention is that it can provide oat protein alternative beverage with similar contents of protein and oil and semi-skimmed milk. This is an advantage compared to commercial oat milk beverages that contain lower protein content. Indeed, commercial oat milks generally comprise 0.4 to 1.7 g of protein per 100 mL of the oat milk and pea-protein supplemented oat milk (Good karma original plant milk, Good karma foods) comprise 2.1 g of protein per 100 mL of the oat milk.

The oat protein alternative beverage of the invention to milk benefited from a creamy mouthfeel and a satisfying taste.

The color of the oat protein alternative beverage to milk was stable during time and between week 0 and week 5, no significant change of the color was observed.

There are other examples of applications in which oat protein of the invention is of interest, for example the ones needing thermal gelling properties of oat protein. This can be a flan dessert, in which oat protein can be included in the form of oat protein powder ingredient or in the form of the oat milk produced here-above.

### Example 11: Preparation of Texturized Vegetable Protein (TVP) Comprising an Oat Protein Composition of the Invention

The following ingredients were used:
Oat protein Isolate: composition similar to the one of Example 3-3
Pea protein: Nutralys® F85M (Roquette)
Internal pea fiber: Pea Fiber 150M (Roquette)

The proteins and pea fiber were mixed using a planetary mixer, Hobart A200, mixed for 10 minutes with a paddle mixer at speed 1

| Ingredients | Quantities (%) |
|---|---|
| Nutralys F85M | 66.5 |
| Oat protein Isolate | 15 |
| Pea Fiber I50M | 18.50 |

The formulas were calculated to have a pea-to-oat protein ratio of 82.5 to 17.5 and a total protein content of 65.7%.

The mixes were extruded using a Coperion ZSK25 corotating twin screw extruder with an L/D=40 and a single circular 2.8 mm diameter die.

Water was adjusted during the extrusion process to reach same approximate target density.

The processing parameters are reported below.

Trial number

| Barrel temp | Trial 1 | Trial 2 |
|---|---|---|
| 2 | 30 | 30 |
| 3 | 30 | 30 |
| 4 | 30 | 30 |
| 5 | 30 | 30 |
| 6 | 110 | 110 |
| 7 | 135 | 135 |
| 8 | 135 | 135 |
| 9 | 96 | 96 |
| Prod. Temp ° C. | 130 | 134 |
| Pressure psi | 700 | 730 |
| Torque % | 27 | 31 |
| Power Kw | 4 | 4 |
| screw speed RPM | 900 | 750 |
| Cutter speed RPM | 900 | 900 |
| Dry feed g/min | 266 | 266 |
| water g/min | 40 | 35 |
| Hydration | 15.0% | |
| % water | 20.0% | |
| SME KJ/Kg | 873.52 | |

Results:

Density was measured using a 1 L graduated cylinder, the cylinder was tared filled to the top with the texturized protein and weighed.

The water absorption was measured with the following method, 20 grams of TVP was weighed in a beaker, 200 grams of distilled water at room temperature was added and the TVP stirred. The sample was stirred again at 10 and 20 minutes. After 30 minutes the water was removed using a sieve and the rehydrated TVP was let stand on the sieve for 5 minutes to have all the free water drain out. The hydrated TVP was then weighed and the water absorption capacity calculated as WAC (expressed in g water/g TVP)=(hydrated weight−20)/20

| Trial number | Density dry | WAC |
|---|---|---|
| Trial 1 | 132 | 2.91 |
| Trial 2 | 108 | 2.48 |

Sensory Evaluation of Rehydrated TVP

TVP samples were rehydrated using the same procedure used to measure water absorption capacity and evaluated for hardness. The three samples were made under the same extrusion screw profile and screw speed.

The samples were evaluated by three scientists using a free form format where the scientists tasted the samples. The samples had soft texture.

1. An oat protein composition having a protein content higher than 50% wherein:
the deamidation degree of the protein is below 40%,
the protein is not substantially chemically substituted,
the solubility of the composition at pH 7 is above 50%,
and the molecular weight Mw of the protein composition is above 25000 g/mol, preferably above 30000 g/mol.

2. The oat protein composition of claim 1 the molecular weight Mw of the protein composition is above 40000 g/mol, for example more than 45000 g/mol or more than 50000 g/mol.

3. The oat protein composition of claim 1 characterized in that composition comprises, based on the total weight of proteins in the composition:
from 0.5 to 30% of proteins having a molecular weight of 300000 g/mol and more, advantageously from 5 to 15%,
from 30 to 75% of proteins having a molecular weight of between 50000 g/mol and 300000 g/mol, advantageously from 45 to 65%,
from 10 to 50% of proteins having a molecular weight of between 10000 g/mol and 50000 g/mol, advantageously from 25 to 45%,
from 0.5 to 20% of proteins having a molecular weight of 10000 g/mol and less, advantageously from 1 to 10%,
the sum making 100%.

4. The oat protein composition of claim 1 wherein the deamidation degree of the protein is below 20%, for example below 15%, or below 10%, or below 5% or around 0%.

5. The oat protein composition of claim 1 wherein the solubility of the composition at pH 7 is above 60%, more advantageously above 70%, preferably above 75%, more preferably above 80%.

6. The oat protein composition of claim 1 wherein the composition contains more than 70% by weight of protein on dry matter based on the total dry weight of the oat protein composition, preferably more than 80% by weight.

7. A process of manufacturing an oat protein composition having a solubility above 50% at pH 7 comprising:
a step of providing a suspension of an oat proteic product, the oat proteic product having a protein content higher than 50% and a molecular weight Mw above 30000 g/mol,
a treatment step of this oat proteic product suspension, the treatment being chosen from an ultrasonication treatment step and a heat treatment step wherein, in the case of the heat treatment of the suspension:
the pH of the suspension is between 8 and 11,
the temperature of the suspension is between 120 and 180° C.
wherein no enzymatic treatment step using endoprotease is done after the treatment step of the oat proteic product suspension.

8. The process of claim 7 wherein the heat treatment step has a duration of 1 to 600 s, advantageously of 2 to 60 s, more advantageously of 5 to 30 s, for example between 10 and 20 s and/or wherein the temperature of the suspension during heat treatment is between 130 and 170° C., advantageously between 135 to 165° C., preferably between 145 and 160° C.

9. The process of claim 7 wherein the pH of the suspension is from 8.2 to 10.5, advantageously from 9.0 to 10.0.

10. The process of claim 7 wherein the treatment is an ultrasonication treatment step.

11. The process of claim 10 wherein the suspension has a pH from 4 to 11, advantageously from 6 to 11, preferably from 7 to 10, more preferably from 7 to 8.5 during the ultrasonication step.

12. The process of claim 10 wherein the total energy input delivered per gram of oat proteic product is from 5 to 200 kJ/g, preferably from 7 to 100 kJ/g, for example from 10 to 80 kJ/g.

13. The process of claim 7 wherein the process further comprises a step of drying the oat protein composition, preferably a spray drying step.

14. The process of claim 7 wherein the oat proteic product provided has a deamidation degree of the protein below 40% and is not substantially chemically substituted.

15. A food or beverage comprising the oat protein composition of claim 1.
